(19) United States
(12) Patent Application Publication
(10) Pub. No.: US 2007/0034214 A1
(43) Pub. Date: Feb. 15, 2007

(54) Method to affect the development of autism spectrum disorders

(76) Inventors: Michael J. Dochniak, (US); Melody B. Dochniak, (US)

Correspondence Address: Michael J. Dochniak, P.O. Box 296, Hugo, MN, 55038, US

(21) Appl. No.: 11/201,807

(22) Filed: Aug. 12, 2005

(51) Int. Cl. A61B 19/00; A61K 39/00

(52) U.S. Cl. 128/898; 424/184.1

(57) ABSTRACT

The invention discloses a method that may affect the development of autism spectrum disorders. The method can be used to substantially reduce mammalian immunoglobulin E (IgE) mediated reaction antibodies that are induced by the allergens inherent in products formed from natural rubber latex. The method may be particularly useful as a proactive approach to affect developmental, learning and behavioral disabilities in children.

### FIELD OF THE INVENTION

The present invention discloses a method that may affect the development of autism spectrum disorders. Specifically, it relates to a method wherein an immature nervous system is provided an environment that is substantially free of natural rubber latex. More specifically, the method can be used to substantially reduce immunoglobulin E (IgE) mediated reaction antibodies that are induced by the allergens inherent in products formed from natural rubber latex.

### DESCRIPTION OF THE PRIOR ART

Autism spectrum disorders cause severe and pervasive impairment in thinking, feeling, language, and the ability to relate to others. These disorders are usually first diagnosed in early childhood and range from a severe form, called autistic disorder not otherwise specified, to a much milder form, Asperger syndrome. They also include two rare disorders, Rett syndrome and childhood disintegrative disorder (National Institutes of Mental Health).

Several patents directed to the diagnosis and treatment of autism spectrum disorders are described below:

U.S. Pat. No. 6,899,876 (Houston) discloses compositions and methods relating to reduction of symptoms of autism. The patent teaches that there has gone unmet a need to improve methods of treating patients with autism who exhibit the effects of exorphins such as gluteomorphin and casomorphin without requiring the patient to adhere to difficult dietary restrictions. Similarly, there has gone unmet a need to protect autistic patients from inadvertent exposure to gluten and casein, typically in the form of dairy products and wheat products.

U.S. Pat. No. 6,790,825 (Beck, et al.) discloses a method of using secretin and compositions made therefrom for the treatment of autism and other neurological, behavioral and immunological disorders. The patent teaches that secretin has never been linked to autistic spectrum disorders, either as a possible cause or treatment, nor has it been used in the treatment of other neurological and/or immunological disorders.

U.S. Pat. No. 6,783,757 (Brudnak) discloses a composition and method for increasing exorphin catabolism to treat autism. The patent teaches that a need thus exists for a more effective manner of breaking down exorphin in the stomach and the intestinal tract of an autistic individual. A need also exists to increase expression of dipeptidylpeptidase IV and like compounds from within an autistic individuals own body, i.e., overcoming the apparent down regulation of these or related genes. A need further exists for increasing the bioavailability of minerals that facilitate digestion reactions.

U.S. Pat. No. 6,585,996 (Lonsdale, et al.) discloses lipid-soluble thiamine derivatives in the treatment of autism. The patent teaches that there remains a need in the art for a pharmaceutical treatment offering clinical improvement in the autistic patient.

U.S. Pat. No. 6,552,000 (Van Kammen) discloses anticonvulsant derivatives useful in treating autism. The patent teaches that clinical studies on topiramate have revealed that previously unrecognized pharmacological properties suggest that topiramate will be effective in treating autism.

U.S. Pat. No. 6,410,527 (Coffin) discloses a method for treating autism by administering an effective amount of certain antagonists. The patent teaches that the drug therapy can effectively treat such disorders including somatoform disorders, dissociative disorders, eating disorders, impulse control disorders, and autism.

U.S. Pat. No. 6,362,226 (Phillips, III, et al.) discloses modulation of in vivo glutamine and glycerin levels in the treatment of autism. The patent teaches that there remains significant room for the improvement in the treatment of autism, particularly in children. A treatment approach that is based on observed biochemical abnormalities in autistic patients would be desirable in view of the potentially applicability of such an approach to 75 to 80% of autistic individuals having primary autism in which no underlying cause is found.

U.S. Pat. No. 6,228,582 (Rodier, et al.) discloses genetic polymorphisms which are associated with autism spectrum disorders. The patent teaches that there is an urgent need for a method of reliably identifying subjects with autism. In particular, there is need for a blood test for polymorphisms causing autism spectrum disorders. Families with affected members need to know whether they carry a mutation which could affect future pregnancies. Clinicians need a test as an aid in diagnosis, and researchers would use the test to classify subjects according to the etiology of their disease.

U.S. Pat. No. 5,686,311 (Shaw) discloses diagnosis of autism and treatment therefore. The patent teaches that the diagnosis test of the invention is preferably carried out by analysis of a urine sample for marker compounds and treating an autistic patient by administering an antifungal drug.

U.S. Pat. No. 5,527,825 (Karson, et al.) discloses a method of treating autism with inhibitors of brain nitric oxide synthase. The patent teaches that the prior art is deficient in the lack of effective means of treating dopamine and nitric oxide dysfunctional states.

U.S. Pat. No. 5,405,943 (Comings) discloses procedures for the diagnosis of disorders by genetic tests to identify deletions or defective alleles in the human tryptophan oxygenase gene and cDNA probes for use in such tests. The patent teaches that abnormal serotonin level (too high or too low) may cause autism.

U.S. Pat. No. 5,008,251 (Gruber) discloses a method of treating autism by administering purine nucleoside and purine nucleoside-related analogs which increase extracellular adenosine concentration. The patent teaches novel methods for stabilizing mast cells.

U.S. Pat. No. 4,920,122 (Naruse, et al.) discloses a pharmaceutical composition and method for the treatment of infantile autism. The patent teaches that tetrahydrobiopterin is surprisingly effective in the treatment of autism.

U.S. Patent Application 20050032896 (Neumann) discloses the use of synthetic retinoic acid in the form of 13-cis vitamin A for the treatment of autism. The application teaches that there is a need to provide means for improving the education of special needs subjects with autism to lessen or alleviate the learning obstacle of subjective self-centered absorption.

The patents described above teach compositions and methods (i.e., reactive approach) to treat the symptoms of autism spectrum disorders. There remains a need for a method (i.e., proactive approach) that substantially eliminates the onset of autism spectrum disorders.

It is generally recognized that immune responses to certain chemicals may have an adverse affect on neurological and cognitive development.

A class of chemicals referred to as toxic metals have been shown to have an adverse affect on neurological and cognitive development. For example, metals such as mercury, cadmium, lead, aluminum, and tin can affect chemical synaptic transmission in the brain and in the peripheral nervous system. Government regulations have been imposed to limit the release of toxic metals such as mercury and lead into the environment.

Foodstuff can trigger an immune response. For example, some food proteins (allergens) can trigger IgE mediated reaction antibodies that cause tissue Mast Cells to release histamine. It has been speculated that an excess of histamine may trigger neuropsychiatic disorders in that histamine is a neurotransmitter and neuromodulator. Some processed foods contain labels that warn consumers of possible allergens (e.g., Allergy Information: Manufactured on equipment that also processes peanuts and other tree nuts—May contain peanuts and other tree nuts.

Finally, materials such as dust, pollen, molds, animal dander, and tiny microorganisms called mites have been known to cause an immune response in some individuals. These materials are often called inhalant allergens because they often enter our system through respiration.

Most recently, there has been an epidemic of developmental, learning and behavioral disabilities in children. It is estimated that about seventeen percent of children in the United States of America currently suffer from such disabilities. Although the chemicals, allergens, and materials described above have been known to affect neurological development and/or trigger an immune response, the frequency of exposure to these substances has not substantially increased and thus may not be considered the primary cause for the recent proliferation of developmental, learning and behavioral disabilities in children.

Studies suggest that the apparent rise in the number of children in the United States with Autism is not the results of “Diagnosis Shifting”. Studies indicate that Autism is increasing with successively younger children, particularly those born between 1987 and 1992. (Examining Autism Prevalence, NAAR-funded Research Leads New Study, 23 Mar. 2005.).

A material that has seen a dramatic increase in usage over the last twenty years is natural rubber latex. It is known that proteins present in natural rubber latex can cause allergic reactions. Exposure to these protein-based allergens through inhalation and dermal absorption has been shown to cause sensitization, adverse allergic reactions, and death through anaphylactic shock. For example, health care workers have experienced an increased number of sensitizations and deaths from protective gloves formed from natural rubber latex. It is estimated that approximately five percent of the population can have an adverse allergic reaction when exposed to natural rubber latex; the adversity of such reactions often increases with the frequency, magnitude, and type of exposure. Efforts have been made to deprotenize natural rubber but a significant number of highly atopic individuals remain ultra sensitive to minute quantities of the allergens.

Children are often exposed to natural rubber latex. For example, it is known that nipples for bottles, pacifiers, diapers, balloons, and many toys are formed from natural rubber latex. Additionally, it is known that food allergens can cross-react with the allergens in natural rubber latex to trigger and adverse immune response. Foods that latex allergic individuals frequently demonstrate sensitivity too include bananas, peaches, plums, cherries, melons, celery, carrots, apples, and pears.

Thus, there remains a need for a method (i.e., proactive approach) that can substantially reduce IgE mediated reaction antibodies induced by natural rubber latex, to affect the development of autism spectrum disorders.

### SUMMARY OF THE INVENTION

The invention discloses a method that can be used to substantially reduce IgE mediated reaction antibodies induced by natural rubber latex, which may affect the development of Autism Spectrum Disorders.

The method comprising the steps of:
a) providing at least one individual at conception;
b) providing an environment for said individual wherein the environment is substantially free of materials formed from natural rubber latex; then
c) allowing said individual to develop in said environment for at least about ten years.

Surprisingly, the method described above can be used to substantially reduce IgE mediated reaction antibodies that have been induced by dissimilar allergens that can cross-react with the allergens in natural-rubber-latex.

It is surmised that the inventive method can be used to substantially reduce IgE mediated reaction antibodies that have been induced by the allergens present in natural-rubber-latex. It is further surmised that adult females having a positive IgE response to the allergens in natural rubber latex during pregnancy are more likely to have offspring that experience developmental, learning and behavioral disabilities including autism spectrum disorders. It is still further surmised that a developing fetus has an increased predisposition to an adverse immune response if one or both parents are adversely affected by the allergens in natural rubber latex. It is finally surmised that an immature nervous system having a positive IgE immune response to the allergens in natural rubber latex are more susceptible to food allergens and non-food allergens. The frequency and intensity of the IgE mediated reaction antibodies induced by such dissimilar allergens that cross-react can adversely affect the severity of the autism spectrum disorder.

Other aspects, objects, features and advantages of the present invention would be apparent to one of ordinary skill in the art from the following detailed description illustrating the preferred embodiments of the invention.

### DETAILED DESCRIPTION OF THE INVENTION

The term “biological age” as described herein is defined as the age in years of an individual starting from conception.

The term “children” as described herein is defined as an individual having a biological age less than about twenty-one years.

The term “natural rubber latex” as described herein is defined as a material extracted from trees and bushes including the hevea brasiliensis tree, the sapotaceae tree, and the guayule bush parthenoim argentatum.

The invention discloses a method that can be used to substantially reduce IgE mediated reaction antibodies induced by natural rubber latex, which may affect the development of autism spectrum disorders.

The method comprising the steps of:
a) providing at least one individual at conception;
b) providing an environment for said individual wherein the environment is substantially free of materials formed from natural rubber latex; then
c) allowing said individual to develop in said environment for at least about ten years.

The environment described in the method of the present invention includes the womb, domiciles, schools, market facilities, and day care facilities.

The natural rubber latex described in the method of the present invention can be any product formed from a material extracted from trees and bushes including the hevea brasiliensis tree, the sapotaceae tree, and the guayule bush parthenoim argentatum.

In the inventive method, the individual is allowed to develop in an environment that is substantially free of natural rubber latex for at least about five years, preferably for at least about ten years, and most preferably for at least about twenty-one years. For example, it is generally understood that the critical period for the development of the brain is from about three weeks to about sixteen weeks from conception, although major structures of the brain continue to develop throughout childhood. For example, neuron migration, cell proliferation, and synapse formation are all very active from conception to about three years and the development of cellular insulation around nerve fibers continues for at least about ten years. The immune system develops extensively during childhood as immune memory (the ability to recognize and respond to foreign proteins) is established.

The method of the present invention can be used to substantially reduce IgE mediated reaction antibodies that may be induced by dissimilar allergens (e.g., foodstuff) that can cross-react with the allergens in natural-rubber-latex.

Some foods to which latex allergic individuals frequently demonstrate sensitivity include avocado, banana, chestnut, kiwi, raw potato, tomato, stone fruit (e.g., peach, plum, cherry) hazelnut, melons, celery, carrot, apple, pear, papaya, and almonds. Reactions are less common but have been reported to peanuts, peppers, citrus fruit, coconut, pineapple, mango, fig, passion fruit, condurango fruit, and ugli fruit.

Reactions to many foods have been reported in latex allergic patients. In many cases, researchers have confirmed the presence of cross-reacting proteins with proteins found in latex. It is now thought that many of these allergic proteins are plant defense proteins found widely in the botanical realm.

While food allergy is common in latex allergic individuals, neither the presence nor the distinct food allergies can be predicted for any patient. More severe latex reactions do appear to necessarily increase the risk of food reactions. Initial manifestations of food allergy can be severe and even anaphylactic. Latex allergic patients should have personal epinephrine syringes available at all times for this reason alone.

All products that come into contact with individuals having a biological age of less than about twenty-one years should be reviewed for possible natural rubber latex content. Products with even minute quantities of natural rubber latex should be avoided.

Currently, children are often exposed to natural rubber latex. For example, it is known that baby nipples for bottles, pacifiers, diapers, balloons, and many toys that young children often gum during teething are formed from natural rubber latex. The frequency and intensity of such exposure to natural rubber latex may increase the likelihood of an IgE antibody response and thus adversely affect neurological development increasing adverse cognitive development including autism spectrum disorders.

Currently, the best treatment for natural rubber latex allergy is avoidance. An allergen-specific IgE antibody test can be run to determine if an individual is more likely to have an adverse reaction to natural rubber latex. Elevated IgE antibodies (positive IgE response) usually indicate an allergy and thus exposure to natural rubber latex should be avoided during neurological development.

Examples of sources of exposure to natural rubber latex in the domiciles include carpeting, condoms, baby bottle nipples, pacifiers, balloons, expandable fabric (waistbands), rubber bands, toys, erasers, gloves, and shoe soles.

Examples of sources of exposure to natural rubber latex in schools includes pencil erasers, rubber bands, sporting equipment, clothing, shoes, carpeting, and nurses office medical gloves.

Examples of sources of exposure to natural rubber latex in market facilities include all of the materials previously described. For example, natural rubber latex is used in a variety of consumer products that can be purchased at market facilities. Examples of such facilities include Wal Mart, Sam's Club, Target, Kmart, Sears, Home Depot, Menards, and Cub Foods. The natural rubber latex is often present in the facility structure and/or present as purchasable consumer goods including flooring, containers, building materials, clothing, novelty items, and food packaging.

Examples of sources of exposure to natural rubber latex in day care facilities include nipples on bottles, pacifiers, rubber gloves, toys, diapers, and clothing.

It is speculated that other cognitive abnormalities may be associated with an adverse immune response to natural rubber latex. For example, evidence indicates that approximately fifty percent of children having autism spectrum disorders are classified as mentally retarded. The method described in the present invention may also have an affect on reducing cognitive disabilities such as mental retardation in individuals that are substantially free of autism spectrum disorders.

It is also speculated that many unexplained childhood deaths could be associated to an adverse immune response (i.e., anaphylactic shock) by exposure to natural-rubber-latex based products. For example, sudden infant death syndrome (S.I.D.S.) continues to be a medical mystery in that no conclusive underlying cause has been found. An adverse symptom of anaphylactic shock relates to a sudden and dramatic decrease in blood pressure. It is speculated that because very young children are in a sensitive biological stage of development such a decrease in blood pressure could be lethal. Furthermore, most recently there has been an increased incidence of children having fatal cardiac arrest during strenuous activity. It is speculated that such deaths could be attributed to an adverse immune response to allergens (e.g., natural rubber latex) that are invasive in our everyday lives.

It is further speculated that immunization shots for children could carry the allergen present in natural rubber latex. For example, medicinal vials are often sealed with rubber-septum's that are formed from natural rubber latex (e.g., bungs on medical vials). It is speculated that N.R.L. based allergens could leach from the rubber septum into the medicine. A syringe extracts the contents of the medicinal vial and the allergen-contaminated medicine is then introduced directly into the child's bloodstream.

What is claimed is:

1. A method to affect the development of autism spectrum disorders, said method comprising the steps of:
a. providing at least one individual at conception;
b. providing an environment for said individual wherein the environment is substantially free of materials formed from natural rubber latex; then
c. allowing said individual to develop in said environment for at least about five years.

2. The method as described in claim 1, wherein said individual has a biological age less than about one year.

3. The method as described in claim 1, wherein said environment is a womb.

4. The method as described in claim 1, wherein said environment is a domicile.

5. The method as described in claim 1, wherein said environment is a school.

6. The method as described in claim 1, wherein said environment is a market facility.

7. The method as described in claim 1, wherein said environment is a day care facility.

8. The method as described in claim 1, wherein said latex is from the hevea brasiliensis tree, the sapotaceae tree, and the guayule bush parthenoim argentatum.

9. The method as described in claim 1, wherein said latex includes deproteinized natural rubber latex.

10. The method as described in claim 1, wherein said individual is allowed to develop in said environment for at least about twenty years.
